(19) United States
(12) Patent Application Publication
(10) Pub. No.: US 2004/0198970 A1
(43) Pub. Date: Oct. 7, 2004

(54) Maxp1

(76) Inventors: Geoffrey J. Clark, Gaithersburg, MD (US); Michelle Vos, Boyds, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/489,906

(22) PCT Filed: Sep. 18, 2002
(86) PCT No.: PCT/US02/29643

(51) Int. Cl. C07H 21/04; C12N 9/10

(52) U.S. Cl. 536/23.5; 435/69.1; 435/193; 435/320.1; 435/325

(57) ABSTRACT

An isolated or purified nucleic acid molecule consisting essentially of a nucleotide sequence encoding human Maxp1, a variant human Maxp1, or a fragment of either of the foregoing; an isolated or purified nucleic acid molecule consisting essentially of a nucleotide sequence that is complementary to a nucleotide sequence encoding human Maxp1, a variant human Maxp1, or a fragment of either of the foregoing; a vector comprising such an isolated or purified nucleic acid molecule; a cell comprising such a vector; an isolated or purified polypeptide molecule consisting essentially of an amino acid sequence encoding human Maxp1, a variant human Maxp1, or a fragment of either of the foregoing; a cell line that produces a monoclonal antibody that is specific for an aforementioned isolated or purified polypeptide molecule; and the monoclonal antibody produced by the cell line. The invention also provides methods of diagnosing a cancer or a predisposition to a cancer in a male or female mammal, a method of prognosticating a cancer in a mammal, a method of assessing the effectiveness of treatment of a cancer in a mammal, and a method of treating a mammal prophylactically or therapeutically for a cancer. Further provided by the invention is a composition comprising a carrier and either (i) an above-described isolated or purified nucleic acid molecule or a fragment thereof, (ii) an above-described vector, (iii) an above-described polypeptide molecule or a fragment thereof, or (iv) an inhibitor of human Maxp1.

### FIELD OF THE INVENTION

This invention pertains to human Maxp1 nucleic acids, vectors, host cells, polypeptides, compositions, monoclonal antibodies and cell lines therefor, and the use of human Maxp1 in the diagnosis, prognosis and treatment of cancer, particularly lung cancer, colon cancer, and renal cancer.

### BACKGROUND OF THE INVENTION

The American Cancer Society estimates the lifetime risk that an individual will develop cancer is 1 in 2 for men and 1 in 3 for women. The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these genetic modifications may not require an exposure to environmental factors to become abnormal. Indeed, certain mutations (e.g., insertions, deletions, substitutions, etc.) can be inherited from generation to generation, thereby imparting an individual with a genetic predisposition to develop cancer.

Currently, the survival rates for many cancers are on the rise. One reason for this success is improvement in the detection of cancer at a stage at which treatment can be effective. Indeed, it has been noted that one of the most effective means to survive cancer is to detect its presence as early as possible. According to the American Cancer Society, the relative survival rate for many cancers would increase by about 15% if individuals participated in regular cancer screenings. Therefore, it is becoming increasingly useful to develop novel diagnostic tools to detect the cancer either before it develops or at an as early stage of development as possible.

One popular way of detecting cancer early is to analyze the genetic makeup of an individual to detect the presence of or to measure expression levels of a marker gene(s) related to the cancer. For example, there are various diagnostic methods that analyze a certain gene or a pattern of genes to detect cancers of the breast, tongue, mouth, colon, rectum, cervix, prostate, testis, and skin. Recently, analyzing the activity of certain Ras effector proteins, particularly those proteins which function as tumor suppressors (e.g., RASSF1), has been found to be useful in diagnosing a cancer or a predisposition to a cancer.

Ras proteins are guanine nucleotide-binding proteins that function by alternating between inactive GDP-bound and active GTP-bound forms. Ras activation is mediated by guanine nucleotide exchange factors that stimulate the release of bound GDP and its exchange for GTP. Activity of the Ras-GTP complex is then terminated by GTP hydrolysis, which is stimulated by the interaction of Ras-GTP with GTPase-activating proteins.

Activated Ras is responsible for mediating multiple biological effects, including regulating the flow of mitogenic signals from cell-surface receptors to the internal cell signaling machinery. This process is achieved by various Ras effectors, which are stimulated downstream of Ras activation, either by binding directly to Ras or by associating with a molecule that has been activated by Ras. Thus, Ras stands at the forefront of a number of signaling pathways that are necessary for proper cell function.

Recently, activated Ras has been determined to be involved with a variety of biological phenotypes associated with abnormal cell growth. Indeed, activated Ras has been associated with a loss of contact inhibition (see, Huber et al., Onco gene, 3:245-256 (1988)), resistance to differentiation (see, e.g., Olson et al., Mol. Cell Biol., 7:2104-2111 (1987)), disruption of cytoskeletal architecture (Hall, Annu. Rev. Cell Biol., 10:31-54 (1994)), reduced requirement for growth factors (Andrejauskas et al., EMBO J., 8:2575-2581 (1989)), invasiveness (see, e.g., Gelmann et al., Int. J. Cancer, 50:665-669 (1992)), and tumorigenic transformation (see, e.g., Malumbres et al., Front. Biosci., 3:d887-d912 (1998)). However, Ras also has been associated with cell growth and inhibition. Indeed, activated Ras may also induce senescence (Serrano et al., Cell, 88:593-602 (1997)), differentiation (Bar-Sagi et al., Cell, 42:841-848 (1985)), and apoptosis (see, e.g., Chen et al., Oncogene, 11: 1487-1498 (1995)). Thus, Ras can either drive processes that are normally associated with the acquisition of a transformed phenotype, or processes that promote growth arrest and death.

Although effectors mediating the positive growth effects of Ras have been relatively well characterized, the effectors mediating the negative growth aspects remain poorly defined. Thus, a need remains for the identification of genes and gene products, particularly those involved in Ras signaling pathways, which can be shown to have a strong association with cancer. Such Ras effectors can lead directly to early, sensitive and accurate methods for detecting a cancer or a predisposition to a cancer in a mammal. Moreover, such methods would enable clinicians to monitor the response of a mammal to a particular treatment with greater sensitivity and accuracy. The present invention provides such methods. These and other advantages of the invention, as well as additional inventive features, will be apparent from the description of the invention provided herein.

### BRIEF SUMMARY OF THE INVENTION

The invention provides an isolated or purified nucleic acid molecule consisting essentially of (i) a nucleotide sequence encoding human Maxp1 or a fragment thereof comprising at least 52 contiguous nucleotides, (ii) a nucleotide sequence encoding a variant human Maxp1, or (iii) a nucleotide sequence that is complementary to (i) or (ii), and related vectors, compositions and host cells.

The invention also provides an isolated or purified polypeptide molecule consisting essentially of (i) an amino acid sequence encoding human Maxp1 or a fragment thereof comprising at least 70 contiguous amino acids or (ii) an amino acid sequence encoding a variant human Maxp1 or a fragment thereof comprising at least 70 contiguous amino acids, and related compositions, monoclonal antibodies (mAb), and mAb producing cell lines.

Further provided by the invention is a method of diagnosing a cancer or a predisposition to a cancer in a mammal. The method comprises detecting a mutation in nucleic acid molecule comprising a nucleotide sequence encoding Maxp1, a decreased level of polypeptide molecule comprising an amino acid sequence encoding wild-type Maxp1, or a mutation in a polypeptide molecule comprising an amino acid sequence encoding Maxp1 in a test sample obtained from the mammal. The detection of a mutation in the nucleic acid or polypeptide molecule encoding Maxp1, or the decreased level of wild-type Maxp1 in the test sample is indicative of the cancer or a predisposition to the cancer in the mammal.

The invention further provides a method of prognosticating a cancer in a mammal and a method of assessing the effectiveness of treatment of a cancer in a mammal. In such methods, Maxp1 is a marker for the cancer. These methods comprise measuring the level of Maxp1 in a test sample obtained from the mammal. The level of Maxp1 in the test sample is indicative of the prognosis or the effectiveness of treatment of the cancer in the mammal.

Still further provided by the invention is a method of treating prophylactically or therapeutically a mammal for a cancer. In such a method, the cancer is due to at least one mutation in a nucleic acid molecule comprising a nucleotide sequence encoding Maxp1, a decreased level of a polypeptide molecule comprising an amino acid sequence encoding wild-type Maxp1, or at least one mutation in a polypeptide molecule comprising an amino acid sequence encoding Maxp1. The method comprises administering to the mammal a composition comprising a carrier and (i) a nucleic acid molecule comprising and expressing a nucleotide sequence encoding wild-type Maxp1 or a fragment thereof, (ii) a nucleic acid molecule comprising and expressing a nucleotide sequence encoding a variant Maxp1 or a fragment thereof, (iii) a polypeptide molecule comprising an amino acid sequence encoding wild-type Maxp1 or a fragment thereof, or (iv) a polypeptide molecule comprising an amino acid sequence encoding a variant Maxp1 or a fragment thereof, wherein the composition is administered to the mammal in an amount sufficient to treat prophylactically or therapeutically the mammal for the cancer.

### BRIEF DESCRIPTION OF THE FIGURES

FIG. 1 represents the nucleotide sequence (SEQ ID NO: 1) of human Maxp1 cDNA.

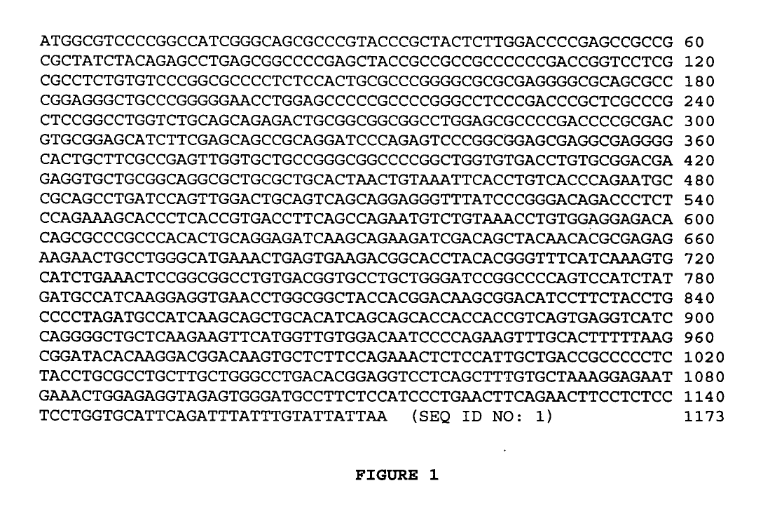

FIG. 2 represents the amino acid sequence (SEQ ID NO: 2) of the polypeptide encoded by the nucleotide sequence of SEQ ID NO: 1.

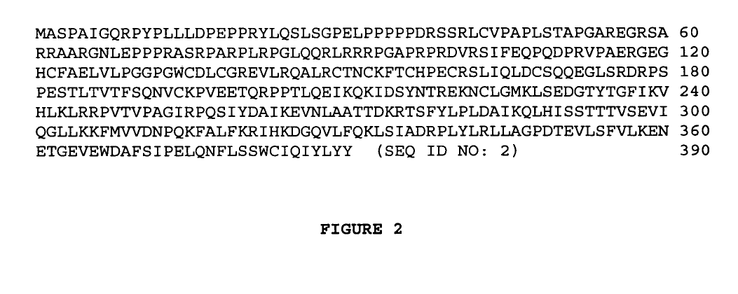

### DETAILED DESCRIPTION OF THE INVENTION

The present invention provides an isolated or purified nucleic acid molecule consisting essentially of a nucleotide sequence encoding human Maxp1 or a fragment thereof comprising at least 52 contiguous nucleotides. Preferably, the isolated or purified nucleic acid molecule (i) encodes the amino acid sequence of SEQ ID NO:2 or a fragment thereof comprising at least 70 contiguous amino acids, (ii) consists essentially of the nucleotide sequence of SEQ ID NO:1 or a fragment thereof comprising at least 52 contiguous nucleotides, (iii) hybridizes under highly stringent conditions to an isolated of purified nucleic acid molecule consisting essentially of the nucleotide sequence that is complementary to SEQ ID NO:1 or a fragment thereof, or (iv) shares 85% or more identity with SEQ ID NO: 1.

While the isolated or purified nucleic acid molecule of the invention consists essentially of a nucleotide sequence encoding human Maxp1 or a fragment thereof comprising at least 52 contiguous nucleotides, larger fragments of human Maxp1 are also contemplated. For example, it is suitable for the isolated or purified nucleic acid molecule of the invention to consist essentially of a nucleotide sequence encoding human Maxp1 or a fragment thereof comprising at least 75 contiguous nucleotides, at least 100 contiguous nucleotides, at least 125 contiguous nucleotides, at least 150 contiguous nucleotides, at least 175 contiguous nucleotides, or even at least 200 contiguous nucleotides. Still larger fragments of human Maxp1 are also contemplated, such as fragments comprising at least 300 contiguous nucleotides, at least 400 contiguous nucleotides, at least 500 contiguous nucleotides, or even at least 600 contiguous nucleotides. Generally, any size fragment is contemplated as long as the fragment comprises contiguous nucleotides spanning 4.4% or more, 10% or more, or even 20% or more of the nucleic acid molecule consisting essentially of the nucleic acid molecule consisting essentially of a nucleotide sequence encoding human Maxp1.

The present invention also provides an isolated or purified polypeptide molecule consisting essentially of an amino acid sequence encoding human Maxp1 or a fragment thereof comprising at least 70 contiguous amino acids, either one of which is optionally glycosylated, amidated, carboxylated, phosphorylated, esterified, N-acylated or converted into an acid addition salt and/or optionally dimerized or polymerized. Preferably, the isolated or purified polypeptide molecule (i) is encoded by the nucleotide sequence of SEQ ID NO:1 or a fragment thereof comprising at least 210 contiguous. nucleotides, (ii) consists essentially of the amino acid sequence of SEQ ID NO:2 or a fragment thereof comprising at least 70 contiguous amino acids, or (iii) shares 84% or more identity with SEQ ID NO:2.

While the isolated or purified polypeptide molecule of the present invention consists essentially of an amino acid sequence encoding human Maxp1 or a fragment thereof comprising at least 70 contiguous amino acids, larger fragments of human Maxp1 are also contemplated. For example, it is suitable for the isolated or purified polypeptide molecule of the invention to consist essentially of an amino acid sequence encoding human Maxp1 or a fragment thereof comprising at least 75 contiguous amino acids, at least 100 contiguous amino acids, at least 125 contiguous amino acids, at least 150 contiguous amino acids, at least 175 contiguous amino acids, or even at least 200 contiguous amino acids. Still larger fragments of human Maxp1 are also contemplated, such as fragments comprising at least 225 contiguous amino acids, at least 250 contiguous amino acids, at least 275 contiguous amino acids, or even at least 300 contiguous amino acids. Generally, any size fragment is contemplated as long as the fragment comprises contiguous amino acids spanning 17.9% or more, 25% or more, or even 30% or more of the polypeptide molecule consisting essentially of an amino acid sequence encoding human Maxp1.

By “isolated” is meant the removal of a nucleic acid or polypeptide molecule from its natural environment. By “purified” is meant that a given nucleic acid or polypeptide molecule, whether one that has been removed from nature or synthesized and/or amplified under laboratory conditions, has been increased in purity, wherein “purity” is a relative term, not “absolute purity”. A “nucleic acid molecule” is intended to encompass a polymer of DNA or RNA, (i.e., a polynucleotide), which can be single-stranded or double-stranded and which can contain non-natural or altered nucleotides. Similarly, a “polypeptide molecule” is intended to encompass a linear sequence of amino acids (i.e., a primary protein structure) but also can include secondary, tertiary, and quaternary protein structures, all of which can contain non-natural or altered amino acids.

With respect to the above isolated or purified nucleic acid molecules, it is preferred that no insertions, deletions, inversions and/or substitutions are present in the nucleic acid molecule. Such a nucleic acid molecule will code for a “non-variant” human Maxp1. However, it is suitable for the above isolated or purified nucleic acid molecules to comprise one or more insertions, deletions, inversion and/or substitutions. Such a nucleic acid molecule will code for a “variant” human Maxp1. In this respect, the present invention provides an isolated or purified nucleic acid molecule consisting essentially of a nucleotide sequence encoding a variant human Maxp1 or a fragment thereof comprising at least 52 contiguous nucleotides. Preferably, the nucleotide sequence encoding such a variant human Maxp1 will have a deletion spanning nucleotides 396 to 1173 (i.e., will comprise nucleotides 1 to 395). In addition, if one or more substitution(s) is present in the isolated or purified nucleic acid molecule encoding the variant human Maxp1, it is preferred that such a substitution(s) results in the substitution of an amino acid of the encoded variant human Maxp1 with another amino acid of approximately equivalent mass, structure and charge.

Similarly, with respect to the above isolated or purified polypeptide molecules, it is preferred that no insertions, deletions, substitutions and/or abnormal post-translational modifications are present in the polypeptide molecule. Such a polypeptide molecule will code for a non-variant human Maxp1. However, it is suitable for the above isolated or purified polypeptide molecules or fragments thereof to comprise one or more insertions, deletions, substitutions and/or abnormal post-translational modifications. Such a polypeptide molecule will code for a variant human Maxp1. In this respect, the present invention provides an isolated or purified polypeptide molecule consisting essentially of an amino acid sequence encoding a variant human Maxp1 or a fragment thereof comprising at least 70 contiguous amino acids. Preferably, the amino acid sequence encoding such a variant human Maxp1 will have a deletion spanning amino acids 132 to 391 (i.e., will comprise amino acids 1 to 131).

Preferably, the variant human Maxp1 will not differ functionally from the corresponding non-variant human Maxp1. For example, any insertions, deletions, inversions and/or substitutions contained within the nucleic acid molecule comprising a nucleotide sequence encoding the variant human Maxp1 will not (i) result in the introduction of a frame-shift mutation, (2) interfere with the ability of the promoter region to direct the transcription of the nucleotide sequence, or (3) interfere with the ability of the corresponding RNA transcript to be translated into a protein. It is also preferred that the one or more substitution(s) result(s) in the substitution of an amino acid with another amino acid of approximately equivalent mass, structure and charge.

Also with respect to the above, “will not differ functionally from” is intended to mean that the variant human Maxp1 will have activity characteristic of the non-variant human Maxp1. However, the variant human Maxp1 can be more or less active than the non-variant human Maxp1 as desired in accordance with the present invention.

Also provided by the invention is a nucleic acid molecule consisting essentially of a nucleotide sequence that is complementary to a nucleotide sequence encoding human Maxp1, a variant human Maxp1 or a fragment of either of the foregoing comprising at least 52 contiguous nucleotides. Preferably, such an isolated or purified nucleic acid molecule (i) is complementary to a nucleotide sequence encoding the amino acid sequence of SEQ ID NO:2, (ii) is complementary to the nucleotide sequence of SEQ ID NO:1 or a fragment thereof comprising at least 52 contiguous nucleotides, (iii) hybridizes under highly stringent conditions to an isolated or purified nucleic acid molecule consisting essentially of SEQ ID NO:1 or a fragment thereof, or (iv) shares 85% or more identity with the nucleotide sequence that is complementary to SEQ ID NO:1.

The phrase “hybridizes to” refers to the selective binding of a single-stranded nucleic acid probe to a single-stranded target DNA or RNA sequence of complementary sequence when the target sequence is present in a preparation of heterogeneous DNA and/or RNA. “Stringent conditions” are sequence-dependent and will be different in different circumstances. Generally, stringent conditions are selected to be about 20° C. lower than the thermal melting point (Tm) for the specific sequence at a defined ionic strength and pH. The Tm is the temperature (under defined ionic strength and pH) at which 50% of the target sequence hybridizes to a perfectly matched probe.

For example, under stringent conditions, as that term is understood by one skilled in the art, hybridization is preferably carried out using a standard hybridization buffer at a temperature ranging from about 50° C. to about 75° C., even more preferably from about 60° C. to about 70° C., and optimally from about 65° C. to about 68° C. Alternately, formamide can be included in the hybridization reaction, and the temperature of hybridization can be reduced to preferably from about 35° C. to about 45° C., even more preferably from about 40° C. to about 45° C., and optimally to about 42° C. Desirably, formamide is included in the hybridization reaction at a concentration of from about 30% to about 50%, preferably from about 35% to about 85%, and optimally at about 40%. Moreover, optionally, the hybridized sequences are washed (if necessary to reduce non-specific binding) under relatively highly stringent conditions, as that term is understood by those skilled in the art. For instance, desirably, the hybridized sequences are washed one or more times using a solution comprising salt and detergent, preferably at a temperature of from about 50° C. to about 75° C., even more preferably at from about 60° C. to about 70° C., and optimally from about 65° C. to about 68° C. Preferably, a salt (e.g., such as sodium chloride) is included in the wash solution at a concentration of from about 0.01 M to about 1.0 M. Optimally, a detergent (e.g., such as sodium dodecyl sulfate) is also included at a concentration of from about 0.01% to about 1.0%.

The following are examples of highly stringent and moderately stringent conditions for a Southern hybridization in aqueous buffers (no formamide) (Sambrook and Russell, Molecular Cloning, 3rd Ed. SCHL Press (2001)):

| | |
|---|---|
| Highly stringent | Moderately Stringent |
| hybridization conditions: | hybridization conditions: |
| 6 × SSC or 6 × SSPE | 6 × SSC or 6 × SSPE |
| 5 × Denhardt's Reagent | 5 × Denhardt's Reagent |
| 1% SDS | 1% SDS |
| 100 μg/ml salmon sperm DNA | 10 μg/ml salmon sperm DNA |
| hybridization at 65-68° C. | hybridization at 58-64° C. |
| Highly stringent | Moderately stringent |
| washing conditions: | washing conditions: |
| 0.1 × SSC/0.1% SDS | 2 × SSC/0.1% SDS |
| washing at 65-68° C. | washing at 58-64° C. |

In view of the above, “highly stringent conditions” preferably allow for up to 15% mismatch, more preferably up to about 12% mismatch, and most preferably up to 10% mismatch (such as 5%, 4%, 3%, 2% or 1%). “Moderately stringent conditions” preferably allow for up to about 40% mismatch, more preferably up to about 30% mismatch, and most preferably up to about 20% mismatch. “Low stringent conditions” preferably allow for up to about 60% mismatch, more preferably up to about 50% mismatch, and most preferably up to about 40% mismatch. With respect to the preceding ranges of mismatch, 1% mismatch corresponds to one degree decrease in the melting temperature. It is generally appreciated that the stringent conditions can be manipulated by adjusting the concentration of formamide in the hybridization reaction. For example, conditions can be rendered more stringent by the addition of increasing amounts of formamide.

The above isolated or purified nucleic acid and polypeptide molecules also can be characterized in terms of “percentage of sequence identity.” In this regard, a given nucleic acid or polypeptide molecule as described above can be compared to a nucleic acid or polypeptide molecule encoding human Maxp1 by optimally aligning the nucleotide or amino acid sequences over a comparison window, wherein the portion of the nucleotide or amino acid sequence in the comparison window may comprise additions or deletions (i.e., gaps) as compared to the reference sequence, which does not comprise additions or deletions, for optimal alignment of the two sequences. The percentage of sequence identity is calculated by determining the number of positions at which the identical nucleotide or amino acid occurs in both sequences, i.e., the number of matched positions, dividing the number of matched positions by the total number of positions in the window of comparison, and multiplying the result by 100 to yield the percentage of sequence identity. Optimal alignment of sequences for comparison may be conducted by computerized implementations of known algorithms (e.g., GAP, BESTFIT, FAGTA, and TFASTA in the Wisconsin Genetics Software Package, Genetics Computer Group (GCG), 575 Science Dr., Madison, Wis.; BlastN and BlastP available from the National Center for Biotechnology Information, Bethesda, Md.; or ClustalW available from the European Bioinformatics Institute, Cambridgeshire, UK), or by inspection. Generally, the isolated of purified nucleic acid molecule consists essentially of a nucleotide sequence which shares 85% or more identity with SEQ ID NO:1, and the isolated or purified polypeptide molecule consists essentially of an amino acid sequence which shares 84% or more identity with SEQ ID NO:2. It will be understood, however, that the percentage of sequence identity may vary slightly when using the different computerized programs since they implement different algorithms. The invention is intended to cover such variations but will generally share the percentage of sequence identities above using at least one computerized program and its respective algorithm.

The present invention also provides a vector comprising an above-described isolated or purified nucleic acid molecule. A nucleic acid molecule as described above can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987)).

Constructs of vectors, which are circular or linear, can be prepared to contain an entire nucleic acid molecule as described above or a portion thereof ligated to a replication system functional in a prokaryotic or eukaryotic host cell. Replication systems can be derived from ColE1, 2 mμ plasmid, λ, SV40, bovine papilloma virus, and the like.

In addition to the replication system and the inserted nucleic acid molecule, the construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

Suitable vectors include those designed for propagation and expansion or for expression or both. A preferred cloning vector is selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clonetech, Palo Alto, Calif.). Bacteriophage vectors, such as λGT10, λGT 11, λZapII (Stratagene), λ EMBL4, and λ NM1149, also can be used. Examples of plant expression vectors include pB1101, pB1101.2, pB1101.3, pB1121 and pBIN19 (Clonetech, Palo Alto, Calif.). Examples of animal expression vectors include pEUK-C1, pMAM and pMAMneo (Clonetech).

An expression vector can comprise a native or nonnative regulatory sequence operably linked to an isolated or purified nucleic acid molecule as described above. If more than one nucleic acid sequence is included in the nucleic acid molecule, each sequence can be operably linked to its own regulatory sequence. The “regulatory sequence” is typically a promoter sequence or promoter-enhancer combination, which facilitates the efficient transcription and translation of the nucleic acid to which it is operably linked. The regulatory sequence can, for example, be a mammalian or viral promoter, such as a constitutive or inducible promoter. Exemplary viral promoters which function constitutively in eukaryotic cells include, for example, promoters from the simian virus, papilloma virus, adenovirus, human immunodeficiency virus, Rous sarcoma virus, cytomegalovirus, Moloney leukemia virus and other retroviruses, and Herpes simplex virus. Other constitutive promoters are known to those of ordinary skill in the art. The promoters useful as regulatory sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art and can be used in the context of the invention, when desired. The selection of promoters, e.g., strong, weak, inducible, tissue-specific and developmental-specific, is within the skill in the art. Similarly, the combining of a nucleic acid molecule as described above with a promoter is also within the skill in the art.

The term “operably linked” as used herein can be defined when a nucleic acid molecule and the regulatory sequence are covalently linked in such a way as to place the expression of the nucleic acid coding sequence under the influence or control of the regulatory sequence. Thus, a regulatory sequence would be operably linked to a nucleic acid molecule if the regulatory sequence were capable of effecting transcription of that nucleic acid molecule such that the resulting transcript is translated into the desired protein or polypeptide.

The present invention provides a cell (i.e., a host cell) comprising an isolated or purified nucleic acid molecule or a vector as described above. Examples of host cells include, but are not limited to, a human cell, a human cell line, E. coli, B. subtilis, P. aerugenosa, S. cerevisiae, and N. crassa. E. coli, in particular E. coli TB-1, TG-2, DH5α, XL-Blue MRF′ (Stratagene), SA2821 and Y1090 are preferred hosts.

Any appropriate expression vector (e.g., as described in Pouwels et al., Cloning Vectors: A Laboratory Manual, Elsevior, N.Y., (1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology, 6, 47 (1988)), and established cell lines such as the COS-7, C127, 3T3, CHO, HeLa, BHK cell line, and the like. The ordinarily skilled artisan is, of course, aware that the choice of expression host has ramifications for the type of polypeptide produced. For instance, the glycosylation of polypeptides produced in yeast or mammalian cells (e.g., COS-7 cells) will differ from that of polypeptides produced in bacterial cells such as Escherichia coli.

If desired, the polypeptide molecules of the invention (including variant polypeptide molecules) can be modified, for instance, by glycosylation, amidation, carboxylation, or phosphorylation, or by the creation of acid addition salts, amides, esters, in particular C-terminal esters, and N-acyl derivatives of the polypeptide molecules of the invention. The polypeptide molecules also can be dimerized or polymerized. Moreover, the polypeptide molecules can be modified to create polypeptide derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N— or C-terminus.

Cell lines producing monoclonal antibodies also are contemplated in the invention. Such “hybridoma cell lines” desirably produce a monoclonal antibody that is specific for human Maxp1. Typically, the monoclonal antibody will be specific for an epitope of an isolated or purified polypeptide molecule encoding human Maxp1. Methods of making hybridomas are known in the art (see, e.g., Roitt I., Immunology, 4^{th} Ed., Mosby, N.Y. (1996)). Thus, the present invention also provides a monoclonal antibody produced by the hybridoma cell line. Such monoclonal antibodies are typically employed for diagnostic applications as they are described herein.

Maxp1 exhibits all the basic biological and biochemical characteristics of a Ras effector, i.e., a protein which directly mediates the biological effects of Ras. However, unlike most previously described Ras effectors, Maxp1 serves to inhibit cell growth. This growth inhibition is enhanced in the presence of an activated form of Ras, and reduced in the presence of a dominant negative form of Ras. Thus, Ras appears to regulate the growth inhibitory properties of Maxp1. Maxp1 serves to connect Ras to numerous signal transduction pathways, including the CREB pathway. Maxp1 is, however, a relatively poor activator of NFkB and it is possible that activation of CREB in the absence of NFkB activation promotes apoptotic cell death (Saeki et al, Biochem J. 1999 Oct 1;343 Pt 1:249-55 (1999)). Maxp1 expression is lost or severely reduced in many lung, breast and colon tumors and tumor cell lines. Moreover, the human gene has been mapped to 1q32.1-2, a site which has been described as the location of an unknown tumor suppressor in kidney tumors (Steiner et al. Cancer Res, 56:50044-5046 (1996)). Thus, Maxp1appears to be a Ras effector which is also a tumor suppressor. Accordingly, the invention provides a method of diagnosing a mammal with a cancer or a predisposition to a cancer. The method comprises detecting either (i) a mutation in a nucleic acid molecule comprising a nucleotide sequence encoding Maxp1, (ii) a decreased level of a polypeptide molecule comprising an amino acid sequence encoding wild-type Maxp1, or (iii) a mutation in a polypeptide molecule comprising an amino acid sequence encoding Maxp1 in a test sample obtained from the mammal. In such a method, the detection of (i), (ii), or (iii) in the test sample is indicative of the cancer or a predisposition to the cancer in the mammal. Preferably, the nucleic acid molecule comprising the nucleotide sequence encoding Maxp1 comprises SEQ ID NO:1 and the polypeptide molecule comprising the amino acid encoding Maxp1 comprises SEQ ID NO:2. Down-regulation can be due to promoter methylation.

The test sample used in conjunction with the invention can be any of those typically used in the art and will vary depending on the condition of the mammal (i.e., whether or not a cancer has developed in the mammal). For example, the test sample can be saliva, tissue or blood. Typically, the tissue is metastatic (e.g., cancerous) and is obtained by means of a biopsy. Such tissue can include bone marrow, lymph nodes, skin, and any organ that may develop cancerous cells. Preferably, however, the test sample is one which is least invasive to the mammal, such as a saliva or blood sample.

A number of assays are contemplated for use in analyzing a given test sample of the present invention. As used herein, the term “assay” can be defined as any quantitative or qualitative analysis of a nucleic acid or polypeptide molecule that is known in the art. A variety of these assays are contemplated for use in the invention, many of which are described in Sambrook et al., Molecular Cloning: A Laboratory Manual, 2^{nd} Ed., Cold Spring Harbor Press, Cold Spring Harbor, N.Y., (1989). Microarrays, such as those described in U.S. Pat. Nos. 6,197,506 and 6,040,138, also can be used to detect and quantify Maxp1. It will be understood that the type of assay used will depend on whether a nucleic acid or polypeptide molecule is being assayed for and whether the detection or quantification of the nucleic acid or polypeptide molecule is sought.

When a nucleic acid molecule encoding a nucleotide sequence encoding Maxp1 is assayed for, various assays can be used to detect or to measure the level of Maxp1 in a given test sample. For example, when only the detection of Maxp1 or the identification of a mutation in Maxp1 is necessary to diagnose effectively the cancer or a predisposition to the cancer, assays including PCR and microarray analysis can be used. In certain embodiments it may be necessary to detect the quantity of Maxp1 present. In such instances, it will be advantageous to use various hybridization techniques known in the art that can effectively measure the level of Maxp1 in a test sample. When Maxp1 comprises DNA, such hybridization techniques can include, for example, Southern hybridization (i.e., a Southern blot), in situ hybridization and microarray analysis. Similarly, when Maxp1 comprises RNA, Northern hybridization (i.e., a Northern blot), in situ hybridization and microarray analysis are contemplated.

It will be understood that, in such assays, a nucleotide sequence that specifically binds to or associates with a nucleic acid molecule comprising a nucleotide sequence encoding Maxp1, whether DNA or RNA, can be attached to a label for determining hybridization. A wide variety of appropriate labels are known in the art, including fluorescent, radioactive, and enzymatic labels as well as ligands, such as avidin/biotin, which are capable of being detected. Preferably, a fluorescent label or an enzyme tag, such as urease, alkaline phosphatase or peroxidase, is used instead of a radioactive or other environmentally undesirable label. In the case of enzyme tags, colorimetric indicator substrates are known which can be employed to provide a detection means visible to the human eye or spectrophotometrically to identify specific hybridization with complementary Maxp1 nucleic acid-containing samples.

When a nucleic acid molecule comprising a nucleotide sequence encoding Maxp1 is amplified in the context of a diagnostic application, the nucleic acid used as a template for amplification is isolated from cells contained in the test sample, according to standard methodologies (see, e.g., Sambrook et al., (1989), supra). The nucleic acid can be genomic DNA or fractionated or whole cell RNA. Where RNA is used, it can be desirable to convert the RNA to cDNA.

In a typical amplification procedure, pairs of primers that selectively hybridize to nucleic acids corresponding to Maxp1 are contacted with the nucleic acid under conditions that permit selective hybridization. Once hybridized, the nucleic acid-primer complex is contacted with one or more enzymes that facilitate template-dependent nucleic acid synthesis. Multiple rounds of amplification, also referred to as “cycles,” are conducted until a sufficient amount of amplification product is produced.

Various template-dependent processes are available to amplify human Maxp1 present in a given test sample. As with the various assays, a number of these processes are described in Sambrook et al. (1989), supra. One of the best-known amplification methods is the polymerase chain reaction (PCR). Similarly, a reverse transcriptase PCR (RT-PCR) can be used when it is desired to convert mRNA into cDNA. Alternative methods for reverse transcription utilize thermostable DNA polymerases and are described in WO 90/07641, for example.

Other methods for amplification include the ligase chain reaction (LCR), which is disclosed in U.S. Pat. No. 4,883,750; isothermal amplification, in which restriction endonucleases and ligases are used to achieve the amplification of target molecules that contain nucleotide 5′-[alpha-thio]-triphosphates in one strand (Walker et al., Proc. Natl Acad. Sci. USA 89: 392-396 (1992)); strand displacement amplification (SDA), which involves multiple rounds of strand displacement and synthesis, i.e., nick translation; and repair chain reaction (RCR), which involves annealing several probes throughout a region targeted for amplification, followed by a repair reaction in which only two of the four bases are present. The other two bases can be added as biotinylated derivatives for easy detection. Target-specific sequences also can be detected using a cyclic probe reaction (CPR). In CPR, a probe having 3′ and 5′ sequences of non-specific DNA and a middle sequence of specific RNA is hybridized to DNA, which is present in a sample. Upon hybridization, the reaction is treated with RNase H, and the products of the probe are identified as distinctive products, which are released after digestion. The original template is annealed to another cycling probe and the reaction is repeated. A number of other amplification processes are contemplated; however, the invention is not limited as to which method is used.

Following amplification of Maxp1, it can be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al. (1989), supra.

Alternatively, chromatographic techniques can be employed to effect separation. There are many kinds of chromatography which can be used in the context of the present inventive methods e.g., adsorption, partition, ion-exchange and molecular sieve, and many specialized techniques for using them including column, paper, thin-layer and gas chromatography (Freifelder, Physical Biochemistry Applications to Biochemistry and Molecular Biology, 2^{nd} Ed., Wm. Freeman and Co., New York, N.Y. (1982)).

Amplification products must be visualized in order to confirm amplification of the Maxp1 sequence. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified Maxp1 sequence. The probe preferably is conjugated to a chromophore but may be radiolabeled. In another embodiment, the probe is conjugated to a binding partner, such as an antibody or biotin, where the other member of the binding pair carries a detectable moiety (i.e., a label).

One example of the foregoing is described in U.S. Pat. No. 5,279,721, which discloses an apparatus and method for the automated electrophoresis and transfer of nucleic acids. The apparatus permits electrophoresis and blotting without external manipulation of the gel and is ideally suited to carrying out methods according to the present invention.

It will be understood that the probes described above are limited in as much as any nucleic acid molecule comprising a nucleotide sequence can be used as long as the nucleic acid molecule comprising the nucleotide sequence is hybridizable to nucleic acid molecules comprising a nucleotide sequence encoding Maxp1 or a fragment thereof. For example, a nucleic acid of partial sequence can be used to quantify the expression of a structurally related gene or the full-length genomic or cDNA clone from which it is derived.

When a polypeptide molecule comprising an amino acid sequence encoding Maxp1 is assayed, various assays (i.e., immunobinding assays) are contemplated to either detect or to measure the level of Maxp1 in a given test sample. In such embodiments, Maxp1, or an antibody able to recognize antibodies that are specific for Maxp1 (i.e., an anti-idiotypic antibody), can be employed to detect antibodies having reactivity therewith, or, alternatively, antibodies can be prepared and employed to detect Maxp1 or an anti-idiotypic antibody thereof. The steps of various useful immunodetection assays have been described in Nakamura et al., Handbook of Experimental Immunology (4^{th} Ed.), Wol. 1, Chapter 27, Blackwell Scientific Publ., Oxford (1987); Nakamura et al., Enzyme Immunoassays: Heterogenous and Homogenous Systems, Chapter 27 (1987) and include Western hybridization (i.e., Western blots), immunoaffinity purification, immunoaffinity detection, enzyme-linked immunosorbent assay (e.g., an ELISA), and radioimmunoassay. A microarray also can be used to detect or measure the levels of Maxp1.

In general, the immunobinding assays involve obtaining a test sample suspected of containing a polypeptide molecule comprising an amino acid sequence encoding Maxp1 or an antibody corresponding to human Maxp1, and contacting the test sample with an antibody in accordance with the present invention, as the case may be, under conditions effective to allow the formation of immunocomplexes. Indeed, a mammal can be diagnosed with a cancer or a predisposition to a cancer by either detecting or quantifying the levels of a polypeptide molecule comprising an amino acid sequence encoding Maxp1, an antibody that recognizes Maxp1, or an antibody that recognizes an antibody that is specific for Maxp1.

Any suitable antibody can be used in conjunction with the present invention. Typically, the antibody is specific for Maxp1, however, the antibody can recognize other antibodies (i.e., an anti-idiotypic antibody) present in a test sample that bind to Maxp1. In the instance that the antibody is specific for Maxp1, the antibody can be specific for any region (i.e., epitope) within Maxp1. Preferably, the epitope will be located in a Ras association domain, such as a C-terminal portion of a Ras association domain. For example, the epitope can comprise a region spanning amino acids 311-327 of the wild-type human Maxp1 protein. The antibody can be a polyclonal or a monoclonal antibody and can be identified using methods well known in the art.

The immunobinding assays for use in the present invention include methods for detecting or quantifying the amount of Maxp1 in a test sample, which methods require the detection or quantitation of any immune complexes formed during the binding process. Here, a test sample suspected of containing a polypeptide molecule comprising an amino acid sequence encoding Maxp1 or an antibody that is specific for Maxp1 would be obtained from a mammal and subsequently contacted with an antibody. The detection or the quantification of the amount of immune complexes formed under the specific conditions is then performed.

Contacting the test sample with an antibody that recognizes Maxp1 or an antibody that is specific for Maxp1 under conditions effective and for a period of time sufficient to allow formation of immune complexes (primary immune complexes) is generally a matter of simply adding the antibody to the sample and incubating the mixture for a period of time long enough for the antibodies to form immune complexes with, i.e., to bind to, Maxp1 or an antibody that is specific for Maxp1. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well-known in the art and can be achieved through the application of numerous approaches. These methods are generally based upon the detection of a label or marker, such as any radioactive, fluorescent, biological or enzymatic tags or labels of standard use in the art. U.S. Patents concerning the use of such labels include U.S. Pat. Nos. 3,817,837, 3,850,752, 3,939,350, 3,996,345, 4,277,437, 4,275,149 and 4,366,241. Of course, additional advantages can be realized by using a secondary binding ligand, such as a second antibody or a biotin/avidin ligand binding arrangement, as is known in the art.

Alternatively, the first added component that becomes bound within the primary immune complexes can be detected by means of a second binding ligand that has binding affinity for the first antibody. In these cases, the second binding ligand is, itself, often an antibody, which can be termed a “secondary” antibody. The primary immune complexes are contacted with the labeled, secondary binding ligand, or antibody, under conditions effective and for a period of time sufficient to allow the formation of secondary immune complexes. The secondary immune complexes are then washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the first antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed.

It will be understood that other diagnostic tests can be used in conjunction with the diagnostic tests described herein to enhance further the accuracy of diagnosing a cancer or a predisposition to a cancer in a mammal. For example, a monoclonal antibody which is known to be specific for a cancer can be used in conjunction with the methods of the invention, or the detection of other genetic abnormalities known to be associated with cancer or a predisposition to a cancer can be employed.

The present invention also provides a method of prognosticating a cancer in a mammal, wherein Maxp1 is a marker for the cancer, which method comprises measuring the level of Maxp1 in a test sample obtained from the mammal, wherein the level of Maxp1 in the test sample is indicative of the prognosis of the cancer in the mammal. The level of Maxp1 in the test sample can be measured by comparing the level of Maxp1 in another test sample obtained from the mammal over time in accordance with the methods described above. A decrease in Maxp1 levels from one sample to the next is indicative of growth and/or metastasis of the cancer (i.e., a negative prognosis), whereas an increase or no change in Maxp1 levels from one sample to the next is indicative of halted growth or even reduction of the cancer (i.e., a positive prognosis).

The invention also provides a method of assessing the effectiveness of treatment of a cancer in a mammal, wherein Maxp1 is a marker for the cancer, which method comprises measuring the level of Maxp1 in a test sample obtained from the mammal, wherein the level of Maxp1 in the test sample is indicative of the effectiveness of the treatment of the cancer in the mammal. The level of Maxp1 in the test sample can be measured by comparing the level of Maxp1 in the test sample to the level of Maxp1in another test sample obtained from the mammal over time in accordance with the methods described above. A decrease or no change in Maxp1 levels from one sample to the next is indicative of the treatment being ineffective, whereas an increase in Maxp1 levels from one sample to the next is indicative of the treatment being effective.

As used herein, the term “decreased level” can be defined as detecting Maxp1 in a test sample obtained from a mammal at a level below that which is considered normal. For example, the level of Maxp1 in a test sample is decreased when the copy number of the gene encoding the Maxp1, the mRNA encoding Maxp1, or a polypeptide molecule comprising an amino acid sequence encoding Maxp1 is detected at a level below that which is considered normal. Conversely, the term “increased level” can be defined as detecting Maxp1 in a test sample obtained from a mammal at a level above that which is considered normal. For example, the level of Maxp1 in a test sample is increased when the copy number of the gene encoding the Maxp1, the MRNA encoding Maxp1, or a polypeptide molecule comprising an amino acid sequence encoding Maxp1 is detected at a level above that which is considered normal. “Normal levels” pertain to an already determined range of Maxp1 established from cancer-free mammals of the same species and are generally accepted and recognized in the art.

It has been proposed that Maxp1 mediates a signaling pathway directed by Ras which leads to growth inhibition. Thus, a mutation in Maxp1 which disrupts its normal function or a decreased level of Maxp1 would, in effect, shut down the growth inhibitory effects of Ras, leaving the pro-transformation effector pathways intact. Accordingly, the present invention further provides a method of treating a mammal prophylactically or therapeutically for a cancer by administering to the mammal a composition comprising a carrier and (a) a nucleic acid molecule comprising and expressing a nucleotide sequence encoding wild-type Maxp1 or a fragment thereof, (b) a nucleic acid molecule comprising and expressing a nucleotide sequence encoding a variant Maxp1 or a fragment thereof, (c) a polypeptide molecule comprising an amino acid sequence encoding wild-type Maxp1 or a fragment thereof, or (d) a polypeptide molecule comprising an amino acid sequence encoding a variant Maxp1 or a fragment thereof, wherein the composition is administered to the mammal in an amount sufficient to treat prophylactically or therapeutically the mammal for the cancer. In such instances, the cancer typically is due to (i) at least one mutation in a nucleic acid molecule comprising a nucleotide sequence encoding Maxp1, (ii) a decreased level of a polypeptide molecule comprising an amino acid sequence encoding wild-type Maxp1, or (iii) at least one mutation in a polypeptide molecule comprising an amino acid sequence encoding Maxp1.

A mammal can be diagnosed with, or predisposed to, any cancer utilizing the methods of the invention. Similarly, the method involving prognosticating a mammal for a cancer, assessing the effectiveness of treatment of a cancer, and treating a mammal prophylactically or therapeutically for a cancer can be utilized with any cancer. Preferably, the cancer is lung cancer, including primary lung tumors, colon cancer, or renal cancer. The cancer can be metastatic. Other cancers contemplated in the invention include: anal cancer; bile duct cancer; bladder cancer; bone cancer; brain and spinal chord cancers; breast cancer; cervical cancer; lymphoma; endometrial cancer; esophageal cancer; gallbladder cancer; gastrointestinal cancer; laryngeal cancer; leukemia; liver cancer; multiple myeloma; neuroblastoma; ovarian cancer; pancreatic cancer; prostatic cancer; retinoblastoma; skin cancer (e.g., melanoma and non-melanoma); stomach cancer; testicular cancer; thymus cancer; thyroid cancer; as well as other carcinomas and sarcomas.

In view of the above, the present invention also provides a composition comprising a carrier and either (i) an above-described isolated or purified nucleic acid molecule or a fragment thereof comprising at least 52 nucleotides, (ii) an above-described vector, or (iii) an above-described polypeptide molecule or a fragment thereof comprising at least 70 amino acids. Preferably, the composition comprises a carrier and a nucleic acid molecule comprising a nucleotide sequence encoding a wild-type or variant Maxp1 or a polypeptide molecule comprising an amino acid sequence encoding a wild-type or variant Maxp1. Most preferably, a nucleic acid molecule encoding a wild-type or variant Maxp1 in a recombinant vector is used, as described above. In such embodiments, it is preferred that the polypeptide molecule comprises a deletion spanning amino acids 132 to 391 and that the nucleic acid molecule comprises a deletion spanning nucleotides 396 to 1173. Such a nucleic acid molecule will code for a variant Maxp1 which comprises a deletion spanning amino acids 132 to 391.

The composition can further comprise, or can be conjugated to, a targeting moiety. Preferably, the targeting moiety is an antibody or an antigenically reactive fragment thereof. The antibody or antigenically reactive fragment thereof can be specific for cancer cells expressing Maxp1, and thus increase the affinity of the composition for the cancer cells. Alternatively, the targeting moiety can be a reporter group, including, but not limited to a radiolabel, a fluorescent label, an enzyme (e.g., that catalyzes a calorimetric or fluorometric reaction), a substrate, a solid matrix, or a carrier (e.g., biotin or avidin).

In accordance with the present invention, an antigenically reactive fragment of the antibody (e.g., Fab, Fc, etc.) can be obtained from the antibodies produced as described above, by methods which include digestion with enzymes, such as pepsin or papain, and/or cleavage of disulfide bonds by chemical reduction.

The composition can comprise more than one active ingredient, such as comprising more than one type of molecule of Maxp1. Alternatively, or additionally, the composition can comprise another pharmaceutically active agent or drug. For example, when treating cancer, other anticancer compounds can be used in conjunction with the composition of the present invention and include, but are not limited to, all of the known anticancer compounds approved for marketing in the United States and those that will become approved in the future. See, for example, Table 1 and Table 2 of Boyd, Current Therapy in Oncology, Section 1. Introduction to Cancer Therapy (J. E. Niederhuber, ed.), Chapter 2, by B. C. Decker, Inc., Philadelphia, 1993, pp. 11-22. More particularly, these other anticancer compounds include doxorubicin, bleomycin, vincristine, vinblastine, VP-16, VW-26, cisplatin, carboplatin, procarbazine, and taxol for solid tumors in general; alkylating agents, such as BCNU, CCNU, methyl-CCNU and DTIC, for brain or kidney cancers; and antimetabolites, such as 5-FU and methotrexate, for colon cancer.

The carrier can be any suitable carrier. Preferably, the carrier is a pharmaceutically acceptable carrier. With respect to compositions, the carrier can be any of those conventionally used and is limited only by chemico-physical considerations, such as solubility and lack of reactivity with Maxp1, and by the route of administration. It will be appreciated by one of skill in the art that, in addition to the above-described composition, the compositions of the present inventive methods can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the Maxp1 and one which has no detrimental side effects or toxicity under the conditions of use.

The choice of carrier will be determined in part by the particular molecule of Maxp1 involved, as well as by the particular method used to administer the composition. Accordingly, there are a variety of suitable formulations of the composition of the present invention. The following formulations for oral, aerosol, parenteral, subcutaneous, intravenous, intramuscular, interperitoneal, rectal, and vaginal administration are exemplary and are in no way limiting.

One skilled in the art will appreciate that suitable methods of administering a composition of the invention to a mammal, in particular a human, are available, and, although more than one route can be used to administer a particular compound, a particular route can provide a more immediate and more effective reaction than another route. Accordingly, the herein-described methods are exemplary and are in no way limiting.

The dose administered to a mammal, in particular a human, should be sufficient to treat prophylactically or therapeutically the cancer in the mammal. One skilled in the art will recognize that dosage will depend upon a variety of factors including the strength of the particular composition employed, as well as the age, species, condition, and body weight of the mammal. The size of the dose will also be determined by the route, timing, and frequency of administration as well as the existence, nature, and extent of any adverse side-effects that might accompany the administration of a particular composition and the desired physiological effect.

Suitable doses and dosage regimens can be determined by conventional range-finding techniques known to those of ordinary skill in the art. Generally, a composition is initially administered in smaller dosages, which are less than the optimum dose of the composition. Thereafter, the dosage is increased by small increments until the optimum effect under the circumstances is reached. The present inventive method will typically involve the administration of about 0.1-100 mg of one or more of the compositions described above per kg body weight.

The following example further illustrates the invention but, of course, should not be construed as in any way limiting its scope.

### EXAMPLES

### Example 1

This example demonstrates that Maxp1 inhibits cell growth.

NIH 3T3 cells (ATCC, Rockville, Md.) were grown in 10% calf serum in DMEM (BRL-Life technologies, Gaithersburg, Md.). These cells were then transfected using the calcium phosphate technique (Clark, G. J., Methods in Enzymology, vol. 255, 395-412 (1995)) with 200 ng of pZIPHA vector or pZIPHAmaxp1 (Fiordalisi, J. J. et al., Methods Enzymol 332: 3-36 (2001)) in 60 mm plates. Cell selection experiments were performed in medium supplemented with 0.5 mg/ml G418 (BRL-Life technologies, Gaithersburg, Md.) for 2 weeks. After 2 weeks the cells were then fixed with 10% methanol/acetic acid and 0.5% crystal violet. Similar experiments were performed with the human breast tumor cell line SK-Br-3 and the human lung tumor cell line H-23 (ATCC, Rockville, Md.).

NIH 3T3 cells transfected with pZipNeoHA. Maxp1 failed to produce G418 resistant colonies. Indeed, dramatic reductions in the number of viable cells after 96 hours were observed in dishes transfected with Maxp1. Similar results were obtained with the SKBr-3 and 293-T cells. Occasional colonies did arise after long-term selection, however, these colonies did not express Maxp1. Moreover, it was shown that Ras and Maxp1 synergize to inhibit growth, while dominant negative Ras blocks growth inhibition.

As indicated by the results above, the growth of NIH 3T3 cells was inhibited when Maxp1 was expressed in the cells. These results indicate that Maxp1 is involved in growth inhibition, and, thus, is a potential tumor suppressor gene. In this regard, re-introduction of the genomic form of Maxp1 into a human lung tumor cell line lacking Maxp1 resulted in a significant impairment of the tumorgenicity of the cell line, as measured by the ability to proliferate in soft agar.

### Example 2

This example demonstrates the involvement of Maxp1 in mediating apoptosis.

Transient transfection assays were also performed to define Maxp1 function. 293-T cells (ATCC, Rockville, Md.) were grown in 10% fetal calf serum in DMEM and were transfected using lipofectamine (BRL-Life technologies) with Ras and Maxp1 in the vector pCDNA (Invitrogen , Carlsbad, Calif.). Transfection of Maxp1 inhibited cell growth after 24-96 hours. Co-transfection of Maxp1 and activated Ras increased the growth inhibition, whereas transfection with a dominant negative form of Ras (61/186 mutant, Stacey et al., Mol. Cell. Biol., 11 (8):4053-64 (1991 Aug) reduced Maxp1 growth inhibition. Co-transfection with pCDNABc12 (generous gift, C. Duckett, NCI, Bethesda, Md.) also reduced growth inhibition suggesting that the inhibition was apoptotic in nature.

Moreover, morphologic analysis of the transfected cells showed that the Maxp1 transfectants showed a similar blebbing phenotype to that observed in 293-T cells transfected with pCDNAFAS, a well known inducer of apoptosis (Vos et al., J. Biol. Chem., 275(46):35669-72 (2000 Nov 17).

As indicated by the results above, Maxp1 mediates a Ras-dependent apoptosis. These results indicate that Maxp1 is a novel Ras effector that is involved in a signaling pathway initiated by Ras, which leads to programmed cell death.

### Example 3

This example demonstrates the production of a polyclonal antibody specific for Maxp1 and its use in diagnosing small cell lung cancer.

A polyclonal antibody was developed using a standard protocol (Research Genetics, Huntsville, Ala.). In this respect, the antibody was raised against an internal Maxp1 peptide (i.e., amino acids 311-327), which is located in the C-terminal Ras associated domain of Maxp1. The resulting polyclonal antibody was specific only for Maxp1 and not specific for related proteins, such as RASSF1 and RASSF2. Moreover, the resulting antibody is able to recognize human, mouse and rat Maxp1 proteins.

The polyclonal antibody described above was used to detect Maxp1 expression levels in test samples known to comprise cancerous cells. Specifically, five randomly selected sections (i.e., pathology slides) of small cell lung carcinomas were incorporated in an immunohistochemical analysis for Maxp1 expression. In four of the five sections, Maxp1 expression was not detected. Similar results have been found in most primary lung tumors by immunohistochemistry.

As indicated by the results above, Maxp1 expression was not present in test samples known to comprise cancerous cells. These results indicate that a polyclonal antibody of the invention can be used to diagnose a cancer in a mammal by detecting low levels or lack of expression of Maxp1.

All of the references cited herein, including patents, patent applications, and publications, are hereby incorporated in their entireties by reference.

While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the claims.

What is claimed is:

1. An isolated or purified nucleic acid molecule consisting essentially of a nucleotide sequence encoding human Maxp1 or a fragment thereof comprising at least 52 contiguous nucleotides.

2. The isolated or purified nucleic acid molecule of claim 1, which (i) encodes the amino acid sequence of SEQ ID NO:2 or a fragment thereof comprising at least 70 contiguous amino acids, (ii) consists essentially of the nucleotide sequence of SEQ ID NO:1 or a fragment thereof comprising at least 52 contiguous nucleotides, (iii) hybridizes under highly stringent conditions to an isolated or purified nucleic acid molecule consisting essentially of the nucleotide sequence that is complementary to SEQ ID NO:1 or a fragment thereof, or (iv) shares 85% or more identity with SEQ ID NO:1.

3. An isolated or purified nucleic acid molecule consisting essentially of a nucleotide sequence encoding a variant human Maxp1, which comprises one or more insertions, deletions, inversions and/or substitutions, wherein the variant human Maxp1 encoded by the isolated or purified nucleic acid molecule does not differ functionally from the corresponding non-variant human Maxp1, or a fragment thereof comprising at least 52 contiguous nucleotides.

4. The isolated or purified nucleic acid molecule of claim 3, wherein the nucleotide sequence encodes a variant human Maxp1 which sequence has a deletion spanning nucleotides 396 to 1173.

5. The isolated of purified nucleic acid molecule of claim 3, wherein the one or more substitution(s) result(s) in the substitution of an amino acid of the encoded variant human Maxp1 with another amino acid of approximately equivalent mass, structure and charge.

6. An isolated or purified nucleic acid molecule consisting essentially of a nucleotide sequence that is complementary to either of a nucleotide sequence encoding human Maxp1 or a fragment thereof comprising at least 52 contiguous nucleotides.

7. The isolated of purified nucleic acid molecule of claim 6, which (i) is complementary to a nucleotide sequence encoding the amino acid sequence of SEQ ID NO:2, (ii) is complementary to the nucleotide sequence of SEQ ID NO:1 or a fragment thereof comprising at least 52 contiguous nucleotides, (iii) hybridizes under highly stringent conditions to an isolated of purified nucleic acid molecule consisting essentially of SEQ ID NO:1 or a fragment thereof, or (iv) shares 85% or more identity with the nucleotide sequence that is complementary to SEQ ID NO:1.

8. An isolated or purified nucleic acid molecule consisting essentially of a nucleotide sequence that is complementary to either of a nucleotide sequence encoding a variant human Maxp1 or a fragment thereof comprising at least 52 contiguous nucleotides.

9. A vector comprising the isolated or purified nucleic acid molecule of claim 1.

10. A vector comprising the isolated or purified nucleic acid molecule of claim 2.

11. A vector comprising the isolated or purified nucleic acid molecule of claim 3.

12. A vector comprising the isolated or purified nucleic acid molecule of claim 4.

13. A composition comprising the isolated or purified nucleic acid molecule of claim 1 and a carrier.

14. A composition comprising the isolated or purified nucleic acid molecule of claim 2 and a carrier.

15. A composition comprising the isolated or purified nucleic acid molecule of claim 3 and a carrier.

16. A composition comprising the isolated or purified nucleic acid molecule of claim 4 and a carrier.

17. A composition comprising the vector of claim 9 and a carrier.

18. A composition comprising the vector of claim 10 and a carrier.

19. A composition comprising the vector of claim 11 and a carrier.

20. A composition comprising the vector of claim 12 and a carrier.

21. A cell comprising the vector of claim 9.

22. A cell comprising the vector of claim 10.

23. A cell comprising the vector of claim 11.

24. A cell comprising the vector of claim 12.

25. An isolated or purified polypeptide molecule consisting essentially of an amino acid sequence encoding human Maxp1 or a fragment thereof comprising at least 70 contiguous amino acids, either one of which is optionally glycosylated, amidated, carboxylated, phosphorylated, esterified, N-acylated or converted into an acid addition salt and/or optionally dimerized or polymerized.

26. The isolated or purified polypeptide molecule of claim 25, which (i) is encoded by the nucleotide sequence of SEQ ID NO:1 or a fragment thereof comprising at least 210 contiguous nucleotides, (ii) consists essentially of the amino acid sequence of SEQ ID NO:2 or a fragment thereof comprising at least 70 contiguous amino acids or (iii) shares 84% or more identity with SEQ ID NO: 2.

27. An isolated or purified polypeptide molecule consisting essentially of an amino acid sequence encoding a variant human Maxp1, which comprises one or more insertions, deletions, substitutions and/or abnormal post-translational modifications, wherein the variant human Maxp1 encoded by the isolated or purified polypeptide molecule does not differ functionally from the corresponding non-variant human Maxp1, or a fragment thereof comprising at least 70 contiguous amino acids, either one of which is optionally glycosylated, amidated, carboxylated, phosphorylated, esterified, N-acylated or converted into an acid addition salt and/or optionally dimerized or polymerized.

28. The isolated or purified polypeptide molecule of claim 27, wherein the amino acid sequence encoding the variant human Maxp1 has a deletion spanning amino acids 132 to 391.

29. A composition comprising the isolated or purified polypeptide molecule of claim 25 and a carrier.

30. A composition comprising the isolated or purified polypeptide molecule of claim 26 and a carrier.

31. A composition comprising the isolated or purified polypeptide molecule of claim 27 and a carrier.

32. A composition comprising the isolated or purified polypeptide molecule of claim 28 and a carrier.

33. A cell line that produces a monoclonal antibody that is specific for an epitope of the isolated or purified polypeptide molecule of claim 25, wherein the epitope is in a region other than the region consisting of amino acids 269 to 337.

34. A cell line that produces a monoclonal antibody that is specific for an epitope of the isolated or purified polypeptide molecule of claim 27, wherein the epitope is in a region other than the region consisting of amino acids 269 to 337.

35. The monoclonal antibody produced by the cell line of claim 33.

36. The monoclonal antibody produced by the cell line of claim 34.

37. A method of diagnosing a cancer or a predisposition to a cancer in a mammal, which method comprises detecting either (i) a mutation in a nucleic acid molecule comprising a nucleotide sequence encoding Maxp1, (ii) a decreased level of a polypeptide molecule comprising an amino acid sequence encoding wild-type Maxp1 , or (iii) a mutation in a polypeptide molecule comprising an amino acid sequence encoding Maxp1 in a test sample obtained from the mammal, wherein the detection of (i), (ii), or (iii) in the test sample is indicative of the cancer or a predisposition to the cancer in the mammal.

38. The method of claim 37, wherein the nucleic acid molecule comprising the nucleotide sequence encoding Maxp1 comprises SEQ ID NO:1.

39. The method of claim 37, wherein the polypeptide molecule comprising an amino acid sequence encoding Maxp1 comprises SEQ ID NO:2.

40. A method of prognosticating a cancer in a mammal, wherein Maxp1 is a marker for the cancer, which method comprises measuring the level of Maxp1 in a test sample obtained from the mammal, wherein the level of Maxp1 in the test sample is indicative of the prognosis of the cancer in the mammal.

41. The method of claim 40, wherein the level of Maxp1 in the test sample is measured by comparing the level of Maxp1 in the test sample to the level of Maxp1 in another test sample obtained from the mammal over time, wherein an increase in the level of Maxp1 over time is indicative of a positive prognosis, and a decrease or no change in the level of human Maxp1 over time is indicative of a negative prognosis.

42. A method of assessing the effectiveness of treatment of a cancer in a mammal, wherein Maxp1 is a marker for the cancer, which method comprises measuring the level of Maxp1 in a test sample obtained from the mammal, wherein the level of Maxp1 in the test sample is indicative of the effectiveness of treatment of the cancer in the mammal.

43. The method of claim 42, wherein the level of Maxp1 in the test sample is measured by comparing the level of Maxp1 in the test sample to the level of Maxp1 in another test sample obtained from the mammal over time, wherein an increase or no change in the level of Maxp1 over time is indicative of the treatment being effective, and a decrease in the level of Maxp1 over time is indicative of the treatment being ineffective.

44. A method of treating a mammal prophylactically or therapeutically for cancer, wherein the cancer is due to (i) at least one mutation in a nucleic acid molecule comprising a nucleotide sequence encoding Maxp1, (ii) a decreased level of a polypeptide molecule comprising an amino acid sequence encoding wild-type Maxp1, or (iii) at least one mutation in a polypeptide molecule comprising an amino acid sequence encoding Maxp1, which method comprises administering to the mammal a composition comprising a carrier and (a) a nucleic acid molecule comprising and expressing a nucleotide sequence encoding wild-type Maxp1 or a fragment thereof, (b) a nucleic acid molecule comprising and expressing a nucleotide sequence encoding a variant Maxp1 or a fragment thereof, (c) a polypeptide molecule comprising an amino acid sequence encoding wild-type Maxp1 or a fragment thereof, or (d) a polypeptide molecule comprising an amino acid sequence encoding a variant Maxp1 or a fragment thereof, wherein the composition is administered to the mammal in an amount sufficient to treat prophylactically or therapeutically the mammal for the cancer.

45. The method of claim 44, wherein the composition comprises a carrier and (a) or (b), wherein (a) or (b) is contained within a recombinant vector.

46. The method of claim 44, wherein the composition comprises a carrier and (a), wherein (a) comprises SEQ ID NO:1.

47. The method of claim 44, wherein the composition comprises a carrier and (b), wherein (b) comprises a nucleic acid molecule comprising a nucleotide sequence encoding a variant Maxp1 which has a deletion spanning nucleotides 396 to 1173.

48. The method of claim 44, wherein the composition comprises a carrier and (c), wherein (c) comprises SEQ ID NO:2.

49. The method of claim 44, wherein the composition comprises a carrier and (d), wherein (d) comprises a polypeptide molecule comprising an amino acid sequence encoding a variant human Maxp1 which has a deletion spanning amino acids 132 to 391.


SEQUENCE LISTING (2 sequences)

SEQ ID NO: 1 - DNA, 1173 bp, Homo sapiens
atggcgtccccggccatcgggcagcgcccgtacccgctactcttggaccccgagccgccgcgctatctacagagcctgagcggccccgagctaccgccgccgccccccgaccggtcctcgcgcctctgtgtcccggcgcccctctccactgcgcccggggcgcgcgaggggcgcagcgcccggagggctgcccgggggaacctggagcccccgccccgggcctcccgacccgctcgcccgctccggcctggtctgcagcagagactgcggcggcggcctggagcgccccgaccccgcgacgtgcggagcatcttcgagcagccgcaggatcccagagtcccggcggagcgaggcgaggggcactgcttcgccgagttggtgctgccgggcggccccggctggtgtgacctgtgcggacgagaggtgctgcggcaggcgctgcgctgcactaactgtaaattcacctgtcacccagaatgccgcagcctgatccagttggactgcagtcagcaggagggtttatcccgggacagaccctctccagaaagcaccctcaccgtgaccttcagccagaatgtctgtaaacctgtggaggagacacagcgcccgcccacactgcaggagatcaagcagaagatcgacagctacaacacgcgagagaagaactgcctgggcatgaaactgagtgaagacggcacctacacgggtttcatcaaagtgcatctgaaactccggcggcctgtgacggtgcctgctgggatccggccccagtccatctatgatgccatcaaggaggtgaacctggcggctaccacggacaagcggacatccttctacctgcccctagatgccatcaagcagctgcacatcagcagcaccaccaccgtcagtgaggtcatccaggggctgctcaagaagttcatggttgtggacaatccccagaagtttgcactttttaagcggatacacaaggacggacaagtgctcttccagaaactctccattgctgaccgccccctctacctgcgcctgcttgctgggcctgacacggaggtcctcagctttgtgctaaaggagaatgaaactggagaggtagagtgggatgccttctccatccctgaacttcagaacttcctctcctcctggtgcattcagatttatttgtattattaa

SEQ ID NO: 2 - protein, 390 aa, Homo sapiens
MASPAIGQRPYPLLLDPEPPRYLQSLSGPELPPPPPDRSSRLCVPAPLSTAPGAREGRSARRAARGNLEPPPRASRPARPLRPGLQQRLRRRPGAPRPRDVRSIFEQPQDPRVPAERGEGHCFAELVLPGGPGWCDLCGREVLRQALRCTNCKFTCHPECRSLIQLDCSQQEGLSRDRPSPESTLTVTFSQNVCKPVEETQRPPTLQEIKQKIDSYNTREKNCLGMKLSEDGTYTGFIKVHLKLRRPVTVPAGIRPQSIYDAIKEVNLAATTDKRTSFYLPLDAIKQLHISSTTTVSEVIQGLLKKFMVVDNPQKFALFKRIHKDGQVLFQKLSIADRPLYLRLLAGPDTEVLSFVLKENETGEVEWDAFSIPELQNFLSSWCIQIYLYY